(19) United States
(12) Patent Application Publication
(10) Pub. No.: US 2009/0018431 A1
(43) Pub. Date: Jan. 15, 2009

(54) Method and apparatus for imaging functional processes in the brain

(76) Inventors: Thorsten Feiweier, Poxdorf (DE); Diana Martin, Herzogenaurach (DE); Gunther Platsch, Rothenbach (DE); Sebastian Schmidt, Weisendorf (DE); Kristin Schmiedehausen, Palo Alto, CA (US); Michael Szimtenings, Bonn (DE)

Correspondence Address: HARNESS, DICKEY & PIERCE, P.L.C., P.O.BOX 8910, RESTON, VA, 20195, US

(21) Appl. No.: 12/216,293

(22) Filed: Jul. 2, 2008

(30) Foreign Application Priority Data
Jul. 9, 2007 (DE) 10 2007 031 930.6

(51) Int. Cl. A61B 5/05

(52) U.S. Cl. 600/407

(57) ABSTRACT

A method and an apparatus are disclosed for imaging functional processes in the brain. In order to be able to correlate the results of MEG examinations with anatomical information and metabolic data, a positron emission tomography measurement is recorded in at least one embodiment by at least one radiation detector, a magnetic resonance imaging measurement is recorded by a coil and a radio-frequency antenna device and a magnetoencephalography measurement is recorded by a plurality of magnetic field sensors, the positron emission tomography measurement and the magnetic resonance imaging measurement being substantially undertaken at the same time, so that the records of the positron emission tomography measurement and the magnetic resonance tomography measurement are isocentric. In at least one embodiment, an evaluation apparatus is provided for carrying out a spatial correlation between the magnetoencephalography measurement and the magnetic resonance imaging measurement, so that registration between the magnetoencephalography measurement and the positron emission tomography measurement results.

### PRIORITY STATEMENT

The present application hereby claims priority under 35 U.S.C. §119 on German patent application number DE 10 2007 031 930.6 filed Jul. 9, 2007, the entire contents of which is hereby incorporated herein by reference.

### FIELD

Embodiments of the invention generally relate to a method and/or an apparatus for imaging functional processes in the brain.

### BACKGROUND

Methods and apparatuses of acquiring physiological processes in the brain are used in particular for diagnosing epilepsies. A multiplicity of different methods, some of which are imaging methods, is used in preoperative epilepsy diagnostics in order to localize the epileptogenic focus and to assess the operability; these are, inter alia: magnetic resonance imaging (MRI), computed tomography (CT), single photon emission computed tomography (SPECT), positron emission tomography (PET), surface electroencephalography (surface EEG), invasive EEG, magnetoencephalography (MEG).

However, each of these examination methods taken individually is insufficient to permit a comprehensive diagnosis. Only correlating and combined evaluation of the results of the individual examination method leads to sufficiently precise statements. In this case, assigning functional measurement results to anatomical information is very important. At present, the anatomical information can be acquired with a very high resolution by magnetic resonance imaging (MRI). By way of example, the functional information can be obtained by methods such as positron emission tomography (PET), single photon emission computed tomography, or a magnetoencephalographic examination (MEG) as well.

The assignment of the functional information to the anatomical information can then be carried out via reference points in the two measurements. However, the methods for functional examination often only permit limited recognition of anatomical structures. Thus, it is certainly possible to subsequently carry out registration of MRI and PET (or SPECT) using commercial software. However, registrations of PET results in relation to MRI examinations still have limited precision, depending on the radioactive tracer used. Even PET with fluorodeoxyglucose (FDG) only has a limited resolution of anatomical details in comparison to MRI; some of the other tracers show almost no anatomical structures. To this extent, neither an automatic nor a manual registration of the data records is sufficiently precise in these cases. This is true in particular for functional information obtained by MEG examinations; these can be assigned to the functional PET information only in a very imprecise manner.

However, the exact determination of a seizure focus is required to be able to remove the epilepsy focus in a targeted manner by a neurosurgical procedure, since large-volume resections far into the healthy brain are neither possible nor sensible due to the expected neurological losses (functional losses for the patient such as paralyses, impaired vision . . . ).

More recent, so-called hybrid modalities combine a plurality of imaging methods in one system. An example of this is a combined MRI-PET system, which in addition permits the simultaneous and isocentric data acquisition of MRI and PET data (after calibration by phantoms, as is also the case in SPECT/CT and PET/CT). Due to the isocentric and simultaneous recording of MRI and PET, an exact registration of the two modalities is automatically present. For the purpose of optimization, MRI movement correction algorithms can also be applied to the PET data, which leads to an improved image quality and localization. A partial volume correction of the PET can also be carried out by using the MRI data.

However, a simultaneous and isocentric MRI examination and MEG examination is not possible, since the MRI examination would magnetically influence the MEG examination.

A complete reading can however only be achieved if metabolic or receptor information is available in addition to the MEG and MRI information in the individual areas. And this metabolic information is obtained in particular by PET examinations.

A neurodiagnostic workstation which is part of a hospital archive and communication network and serves for planning epilepsy-related surgical procedures by combining biomedical information from four neuroimaging modalities is disclosed in S. T. C. Wong et al., “Multimodal Image Fusion for Noninvasive Epilepsy Surgery Planning”, IEEE Computer Graphics and Applications 16 (January 1996), 30-8.

DE 101 44 630 A1 discloses a method and an apparatus for visualizing a body-volume. In the method, a synthesized display is calculated from at least two selected diagnostic data records, the data values of the synthesized display being calculated in each case as a mathematical function of at least one data value of each of the selected data records, and the synthesized display being displayed on the display.

### SUMMARY

In at least one embodiment of the invention, results of MEG examinations arte correlated with anatomical information and metabolic data.

The idea of at least one embodiment of the invention is based on correlating the MEG with the PET and with the MRI. If the MRI data record is then used as anatomical reference in the MEG record, then the registration in relation to the PET is also automatically defined thereby and can then be displayed in a fused fashion and read. In other words, a hybrid PET-MRI measurement is carried out by isocentric and simultaneous recordings of PET and MRI. The data from third measurements, which until now were only registered in relation to the MRI, can be correlated with this hybrid measurement and, in particular, exactly in relation to the PET as well. This then permits a reading with an overview of all three measurements.

The method according to at least one embodiment of the invention for imaging functional processes in the brain comprises, inter alia, the following steps: recording a positron emission tomography measurement by at least one radiation detector for acquiring positron-annihilation radiation from an examination space, recording a magnetic resonance imaging measurement by at least a coil for generating a basic magnetic field, a gradient coil for generating a gradient magnetic field in the examination space and a radio-frequency antenna device for sending excitation pulses into the examination space and for receiving magnetic resonance signals from the examination space, and recording a magnetoencephalography measurement by a plurality of magnetic field sensors for acquiring a spatial and temporal change in the brain's magnetic field, and is characterized in that the positron emission tomography measurement and the magnetic resonance imaging measurement are substantially undertaken at the same time, so that the records of the positron emission tomography measurement and the magnetic resonance imaging measurement are isocentric, and a spatial correlation between the magnetoencephalography measurement and the magnetic resonance imaging measurement is undertaken by an evaluation apparatus, so that registration between the magnetoencephalography measurement and the positron emission tomography measurement results.

As a result of the positron emission tomography measurement and the magnetic resonance imaging measurement being substantially undertaken at the same time, any subsequent matching of the two measurement results is dispensed with, the two records being generated synchronously and isocentrically.

In particular, as a further step, the method displays the record of the positron emission tomography measurement, the record of the magnetic resonance imaging measurement and the record of the magnetoencephaldgraphy measurement on a display device at the same time. This results in the advantage of immediately being able to anatomically assign the PET data.

In a further example embodiment, a further step of the method comprises determining first reference points in the record of the magnetic resonance imaging measurement, determining second reference points in the record of the magnetoencephalography measurement and matching the first and second reference points and displaying the record of the positron emission tomography measurement, the record of the magnetic resonance imaging measurement and the record of the magneto-encephalography measurement on the display device, so that the first and second reference points substantially coincide. In this manner, coinciding displays of the measurement results are obtained automatically, so that the PET results as well can be interpreted at once in an overview with the MEG results.

Correspondingly, the apparatus according to at least one embodiment of the invention for imaging functional processes in the brain is provided with: at least one radiation detector for acquiring positron annihilation radiation from the examination space as a record of a positron emission tomography measurement, a coil for generating a basic magnetic field and at least one gradient coil for generating a gradient magnetic field in the examination space and a radio-frequency antenna device for sending excitation pulses into the examination space and for receiving magnetic resonance signals from the examination space as a record of a magnetic resonance imaging measurement, and a plurality of magnetic field sensors for acquiring a spatial and temporal change in the brain's magnetic field as a record of a magnetoencephalography measurement, and is characterized in that the positron emission tomography measurement and the magnetic resonance imaging measurement are substantially undertaken at the same time, so that the records of the positron emission tomography measurement and the magnetic resonance imaging measurement are isocentric, and an evaluation apparatus is provided for carrying out a spatial correlation between the magnetoencephalography measurement and the magnetic resonance imaging measurement, so that registration between the magnetoencephalography measurement and the positron emission tomography measurement results.

In particular, a further feature of at least one embodiment of the invention is that the radiation detector and the at least one gradient coil are arranged coaxially and substantially at the same axial height around the examination space. As a result of this, the PET and MRI measurements can be correlated with one another without any further post-processing.

### BRIEF DESCRIPTION OF THE DRAWINGS

Further features and advantages of the invention emerge from the following description of example embodiments, with reference being made to the attached drawings.

FIG. 1 shows a combined MRI/PET unit according to the prior art in a perspective illustration.

FIG. 2 shows a cross section of the combined MRI/PET unit according to FIG. 1.

FIG. 3 shows the head of a subject with magnetic field sensors applied thereto according to the prior art.

FIG. 4 shows an illustration of the measurement result according to FIG. 3.

FIG. 5 schematically displays an embodiment of the method or apparatus according to an embodiment of the invention for imaging functional processes in the brain.

The drawing is not to scale. Elements which are the same or act in the same manner are provided with the same reference symbols.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected, ” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

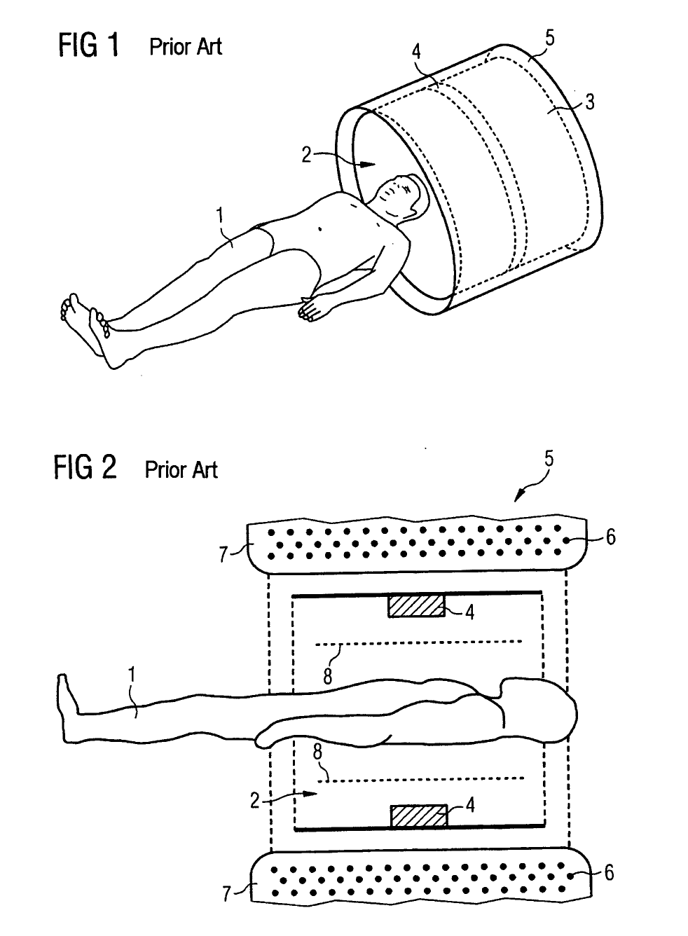

FIG. 1 illustrates the combination of positron emission tomography (PET) and magnetic resonance imaging (MRI). In the case of combined PET and MRI, a subject 1 is brought into an examination space 2. The examination space 2 is directly surrounded by a PET apparatus 3 which comprises a detector device 4. The PET apparatus 3 detects positrons which are released in the body of the subject 1 due to radioactive decay. In order to achieve this, prior to the examination the subject 1 is administered corresponding medicines or compounds (radiopharmaceuticals) in which there is intended a radioactive isotope which accumulates in the tissue in accordance with the bodily function. The positrons released with an initial energy of between 0 eV and a few MeV are scattered in the surrounding tissue and thus decelerate more and more. Once a certain kinetic energy is reached, they can be captured by an electron and annihilate with the latter after 0.1 ns to 150 ns, usually emitting two 511 kev photons with diametrically opposed flight paths. The detector device 4 is generally an arrangement of scintillation crystals (not shown), which are arranged annularly around the examination space. In the scintillation crystals, the photons having an energy of 511 keV (annihilation radiation of the positrons) are converted into light quanta, which in turn are then lead, preferably directly or via optical waveguides (not shown), to photodetectors (not shown) which generate electric output signals depending on the number of light quanta.

In order to be able to assign the examination results of the PET measurement anatomically in the subject 1, the PET apparatus is combined with a MRI apparatus 5. Both apparatuses are explained below on the basis of FIG. 2, which illustrates a configuration of a combined PET and MRI apparatus in cross section. The examination space 2 of the combined PET/MRI apparatus is substantially defined by a gradient coil 6 in a housing 7 and a radio-frequency antenna device 8. The subject 1 is partially located in the examination space 2. The gradient coil 6, which generates a magnetic field in the examination space 2, is arranged outside around the examination space 2. The gradient coil is responsible only for coding spatial information. The polarization or alignment of the spins is carried out by a main magnetic field magnet (not shown), which concentrically surrounds the gradient coil. By means of the magnetic field, the spins of the atomic nuclei in the body of the subject 1 are at least partially aligned, so that the degeneracy of their magnetic quantum number is cancelled. By means of the radio-frequency antenna device 8, transitions between the no-longer degenerate states are induced. The relaxation signals of the transitions are captured by the same radio-frequency antenna device and are passed on to processing electronics (not shown). Subsequently, they are graphically displayed for evaluation.

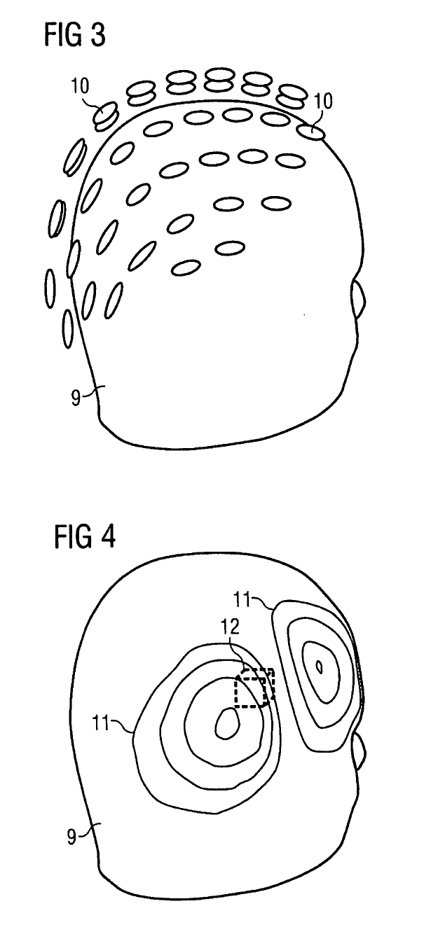

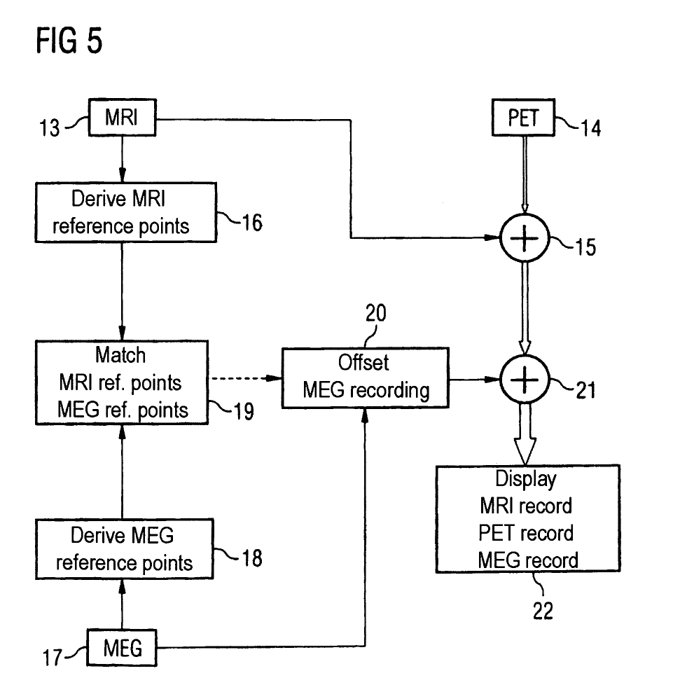

The basic principle of a magnetoencephalography measurement (MEG) is explained on the basis of FIGS. 3 and 4. In the case of an MEG, the magnetic field generated by brainwaves is recorded. Subsequently, the magnetoencephalography measurement is evaluated by certain algorithms, the measurement of the magnetic field taking place outside of the body (non-invasively) and allowing conclusions about current sources in the brain, neuronal processes being associated with current flows of high density. The magnetic field is acquired by magnetic field sensors 10 which are arranged around the head 9 of a subject. By way of example, such a whole head system can have 148 channels with 148 sensors. The sensors are He-cooled SQUIDs (superconducting quantum interference devices), by means of which magnetic fields of 10⁻⁹ Tesla can be measured (for comparison: the Earth's magnetic field has a strength of approximately 10⁻⁴ Tesla). The MEG allows electrophysiological data to be measured directly and with a temporal resolution of less than a millisecond, without this representing a burden for the patient/subject. However, one disadvantage is their low spatial resolution and their initial lack of anatomical assignment. For this reason there is great interest in combining MEG with the highly spatially resolving MRI, for example.

The result of a measurement by the system of sensors 10 in FIG. 3 on a head 9 can be visualized as in FIG. 4. FIG. 4 displays isomagnetic lines 11 on the head 9 which have been derived from the measurement and which lead to identifying a volume 12 in the interior of the head which generates the magnetic field (dipole). In association with an MRI measurement, an actual anatomical structure of the brain in the head 9 of the subject 1 could be associated with the generation of the acquired magnetic field 11 rather than an abstract cube 12.

According to an embodiment of the invention, a method or an apparatus is thus used which is explained in the following on the basis of FIG. 5.

In the step 13, or by the apparatus 13, an MRI measurement is recorded. Simultaneously and at the same location in the examination space 2, a PET measurement is recorded in step 14, or by the apparatus 14. The two measurements are superposed in 15, so that they can be displayed simultaneously on one and the same display and permit the observer to assign functional results from the PET to anatomical results from the MRI.

According to an embodiment of the invention, at least one spatial reference point in the MRI record is fixed at 16 independently of the above processes.

In 17 a MEG measurement is recorded with the aid of which the previously mentioned magnetic fields of the brain are acquired. By analogy with 16, at least one spatial reference point is fixed in the MEG record in 18.

The two (or more) reference points from 16 and 18, respectively, are compared with one another in 19, and a corresponding value is calculated depending on this comparison which is intended to permit the linking of records in the course of the method. This comparison is very complex and involved in detail, since the MEG record and the MRI record can be carried out neither at the same location, which is to say under the same geometric conditions for the subject, nor at the same time. Thus, the reference points in the two records must first of all be defined independently of one another. For this purpose, points should be selected in each record which can also be identified in the respective other record in an easy and spatially precise manner. The structures of the PET are not suited for this purpose since, one the one hand, they are not as precisely resolved, and, on the other hand, they are not accessible on the patient (they are located in the depths of his head).

The comparison result from 19 is then used in order to combine the records from 13 and from 17 in such a way that they virtually lie on top of each other. For this purpose, it can be necessary to displace one of the records with regard to the respective other record. In the embodiment of the invention according to FIG. 5, the MEG record is displaced in 20 in such a way that it can be superposed on the MRI record so that the selected reference points lie on top of each other in the two records.

In 21, the MEG record of 20 is superposed on the two MRI and PET records from 15. Now, all the information is together in one display, namely spatial information of high resolution from the MRI record, first functional information having metabolic data from the PET record, and second functional information having physical data (magnetic dipole due to brainwave activity) from the MEG measurement. This combined information is finally displayed together in 22.

By way of example, with this precise correlation of a reduced glucose metabolism, decreased benzodiazepine receptor density or increased tryptophan ingestion (in the case of tuberous sclerosis) in the PET with the evidence of abnormal magnetic (and hence also electrical activity) activity in the MEG of a particular region of the brain allows increased diagnostic certainty. The exact registration of the results of the two functional methods PET and MEG, which can provide evidence for an epileptogenic focus with a higher certainty than anatomical methods, in relation to the exact anatomical information of the MRI is a prerequisite for planning an operative procedure and thus permits the neurosurgical removal of the epileptogenic focus. The anatomical guiding structures which the neurosurgeon sees and can identify during the procedure can be displayed only in the MRI, and this MRI serves, respectively, as the basis for neuronavigation.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for imaging functional processes in the brain, comprising:
recording a positron emission tomography measurement by at least one radiation detector for acquiring positron-annihilation radiation from an examination space;
recording a magnetic resonance imaging measurement by at least a coil for generating a basic magnetic field, a gradient coil for generating a gradient magnetic field in the examination space and a radio-frequency antenna device for sending excitation pulses into the examination space and for receiving magnetic resonance signals from the examination space; and
recording a magnetoencephalography measurement by a plurality of magnetic field sensors for acquiring a spatial and temporal change in the brain's magnetic field, the positron emission tomography measurement and the magnetic resonance imaging measurement being substantially undertaken at the same time, so that the records of the positron emission tomography measurement and the magnetic resonance imaging measurement are isocentric, and a spatial correlation between the magnetoencephalography measurement and the magnetic resonance imaging measurement being undertaken by an evaluation apparatus, so that registration between the magnetoencephalography measurement and the positron emission tomography measurement results.

2. The method as claimed in claim 1, further comprising:
displaying the record of the positron emission tomography measurement, the record of the magnetic resonance imaging measurement and the record of the magnetoencephalography measurement on a display device at the same Lime.

3. The method as claimed in claim 2, further comprising:
determining first reference points in the record of the magnetic resonance imaging measurement,
determining second reference points in the record of the magnetoencephalography measurement,
matching the first and second reference points, wherein the displaying the record of the positron emission tomography measurement, the record of the magnetic resonance imaging measurement and the record of the magnetoencephalography measurement on the display device, is done so that the first and second reference points substantially coincide.

4. An apparatus for imaging functional processes in the brain, comprising:
at least one radiation detector to acquire positron annihilation radiation from the examination space as a record of a positron emission tomography measurement;
a coil to generate a basic magnetic field and at least one gradient coil to generate a gradient magnetic field in the examination space;
a radio-frequency antenna device to send excitation pulses into the examination space and to receive magnetic resonance signals from the examination space as a record of a magnetic resonance imaging measurement;
a plurality of magnetic field sensors to acquire a spatial and temporal change in the brain's magnetic field as a record of a magnetoencephalography measurement, the positron emission tomography measurement and the magnetic resonance imaging measurement being substantially undertaken at the same time, so that the records of the positron emission tomography measurement and the magnetic resonance imaging measurement are isocentric, and
an evaluation apparatus to carry out a spatial correlation between the magnetoencephalography measurement and the magnetic resonance imaging measurement, so that registration between the magnetoencephalography measurement and the positron emission tomography measurement results.

5. The apparatus as claimed in claim 4, wherein the at least one radiation detector and the at least one gradient coil are arranged coaxially and substantially at the same axial height around the examination space.

6. An apparatus for imaging functional processes in the brain, comprising:
means for acquiring positron annihilation radiation from the examination space as a record of a positron emission tomography measurement;
means for generating a basic magnetic field and means for generating a gradient magnetic field in the examination space;
means for sending excitation pulses into the examination space and for receiving magnetic resonance signals from the examination space as a record of a magnetic resonance imaging measurement;
means for acquiring a spatial and temporal change in the brain's magnetic field as a record of a magnetoencephalography measurement, the positron emission tomography measurement and the magnetic resonance imaging measurement being substantially undertaken at the same time, so that the records of the positron emission tomography measurement and the magnetic resonance imaging measurement are isocentric; and
means for carrying out a spatial correlation between the magnetoencephalography measurement and the magnetic resonance imaging measurement, so that registration between the magnetoencephalography measurement and the positron emission tomography measurement results.

7. The apparatus as claimed in claim 6, wherein the means for acquiring positron annihilation radiation and the means for generating a gradient magnetic field are arranged coaxially and substantially at the same axial height around the examination space.

8. A computer readable medium including program segments for, when executed on a computer device, causing the computer device to implement the method of claim 1.
